(19) United States
(12) Patent Application Publication
(10) Pub. No.: US 2024/0241128 A1
(43) Pub. Date: Jul. 18, 2024

(54) PERIVASCULAR ACCUMULATION OF IMMUNE CELLS IN THE DIAGNOSIS AND TREATMENT OF CANCER

(71) Applicants: NeoGenomics Laboratories, Inc., Fort Myers, FL (US); The University of Sheffield, Sheffield (GB)

(72) Inventors: Claire Lewis, Sheffield (GB); Anna Juncker-Jensen, San Clemente, CA (US); Mate Levente, Nagy, CA (US); Nicholas Matthew Stavrou, Aliso Viejo, CA (US); Mohammed Ridha Moamin, Sheffield (GB); Richard Allen, Chesterfield (GB)

(73) Assignees: NeoGenomics Laboratories, Inc., Fort Myers (GB); The University of Sheffield, Sheffield (GB)

(21) Appl. No.: 18/286,040

(22) PCT Filed: Apr. 8, 2022
(86) PCT No.: PCT/US22/24067

§ 371 Date: Oct. 6, 2023

Related U.S. Application Data
(60) Provisional application No. 63/172,977, filed on Apr. 9, 2021.

(51) Int. Cl. G01N 33/574; G01N 33/569

(52) U.S. Cl. CPC G01N 33/57484; G01N 33/56972; G01N 33/57415; G01N 33/57434

(57) ABSTRACT

Provided herein are methods for determining the chance of a tumor not responding to an anti-cancer therapy (e.g., a T cell-based immunotherapy) based on the presence, density, number, and/or location of certain three-cell structures as described herein. The three-cell structures may comprise a T cell, an immunosuppressive tumor-associated macrophage, and an immunosuppressive regulatory T cell. Such methods may be useful for identifying patients not likely to respond to T cell-based immunotherapy. Also provided herein are methods for determining the prognosis and/or invasiveness of a tumor. The present disclosure also encompasses methods for treating a tumor, as well as kits for performing the methods disclosed herein.

### RELATED APPLICATIONS

This application claims priority under 35 U.S.C. § 119(e) to U.S. Provisional Application, U.S. Ser. No. 63/172,977, filed Apr. 9, 2021, which is incorporated herein by reference.

### BACKGROUND OF THE INVENTION

The spatial organization and density of the immune infiltrate in the tumor microenvironment, referred to as immune contexture, can yield information relevant to prognosis and prediction of response to immunotherapy in cancer. Specifically, a distinct subset of tumor-associated macrophages (TAMs) accumulates around blood vessels in mouse tumors and stimulates tumor angiogenesis and various steps in the metastatic pathway. These perivascular (PV) cells also limit tumor responses to frontline anti-cancer therapies like irradiation, chemotherapy, and anti-vascular agents and dampen anti-tumor immunity by recruiting regulatory T cells (via their release of CCL17) and suppressing the proliferation of T cells (Lewis et al. The Multifaceted Role of Perivascular Macrophages in Tumors. Cancer Cell. 2016, 30, 18-25). However, the presence and phenotype of such PV TAMs, as well as their association with other immune cells in the PV niche, has yet to be investigated in human tumors. Accordingly, there is a need to investigate the associations among these cells, and such information could be useful in the determination of patient responses to anti-tumor therapies and for other therapeutic, prognostic, and diagnostic applications.

### SUMMARY OF THE INVENTION

Using immunofluorescence staining techniques and quantitative image analysis, the present disclosure describes the discovery of a three-cell structure present mainly in the stroma of a tumor (e.g., around blood vessels in tumor rich regions) or around tumor cells. The three-cell structures comprise a T cell, an immunosuppressive tumor-associated macrophage, and an immunosuppressive regulatory T cell. The frequency of these three-cell structures (also referred to throughout the present disclosure as immunosuppressive cell trios (ICTs) and triads), particularly in the perivascular stroma of tumors, may correlate with the efficacy of immunotherapies, such as T cell-based immunotherapies, as the two immunosuppressive cell types are highly likely to suppress the function of T cells as they cross from the vasculature into tumors. The frequency of these three-cell structures may also correlate with the efficacy of immunotherapies mediated by activated T cells such as checkpoint inhibitors.

Accordingly, the present disclosure provides methods for predicting the responsiveness of a tumor (and/or determining the chance of a tumor not responding) to an anti-cancer therapy (e.g., a T cell-based immunotherapy) based on the presence, density, number, and/or location of certain three-cell structures as described herein (i.e., structures composed of a regulatory T cell, a tumor-associated macrophage, and a cytotoxic T cell). Also provided herein are methods for determining the prognosis and/or invasiveness of a tumor. The present disclosure also encompasses methods for treating a tumor, and in particular, for determining the appropriate therapy for successfully treating an individual with cancer. Kits, systems, and compositions for performing the methods disclosed herein are also contemplated by the present disclosure.

In one aspect, the present disclosure provides methods for predicting the responsiveness of a tumor (and/or determining the chance of a tumor not responding) to an anti-cancer therapy, such as immunotherapy, comprising the steps of detecting in a tumor sample the presence of three-cell structures comprising a T cell within approximately 100 μm (e.g., within 90 μm, 80 μm, 70 μm, 60 μm, 50 μm, 40 μm, 30 μm, 20 μm, 15 μm, 10 μm, 5, μm, or less than 5 μm from, or in particular in direct contact with) of an immunosuppressive tumor-associated macrophage and an immunosuppressive regulatory T cell; calculating the density or number of the three-cell structures in the tumor sample; and determining the chance of the tumor not responding to the anti-cancer therapy, such as immunotherapy, wherein the chance of the tumor not responding increases as the density or number of the three-cell structures in the tumor sample increases. In certain embodiments, the anti-cancer therapy is a T cell-based immunotherapy. In some embodiments, the T cells in the three-cell structures may be within 90 μm, 80 μm, 70 μm, 60 μm, 50 μm, 40 μm, 30 μm, 20 μm, 15 μm, 10 μm, 5, μm, or less than 5 μm from, or in direct contact with, the immunosuppressive tumor-associated macrophages and immunosuppressive regulatory T cells, or in direct contact with the immunosuppressive tumor-associated macrophages and immunosuppressive regulatory T cells. In some embodiments, the T cell in the T cell structure may be within 20 μm, 15 μm, 10 μm, 5 μm, or less than 5 μm from (and in particular, in direct contact with) the immunosuppressive tumor-associated macrophage and/or immunosuppressive regulatory T cell. In some embodiments, the immunosuppressive regulatory T cell in the three-cell structures may be within 20 μm, 15 μm, 10 μm, 5 μm, or less than 5 μm from (in particular in direct contact with) the immunosuppressive tumor-associated macrophage and/or T cell. In some embodiments, the immunosuppressive tumor-associated macrophages may be within 20 μm, 15 μm, 10 μm, 5 μm, or less than 5 μm from (in particular in direct contact with) the immunosuppressive regulatory T cell and/or T cell. In some embodiments, the three-cell structures comprise T cells in direct contact with immunosuppressive tumor-associated macrophages and/or regulatory T cells. In some embodiments, the T cells are non-functional CD8+ T cells (i.e., inactive PD1−LAG3−CD8+ T cells and/or exhausted PD1+LAG3+CD8+ T cells). In certain embodiments, the T cells are PD1−LAG3−CD3+CD8+ T cells. In certain embodiments, the tumor-associated macrophages are CD68+, CD68+TIM3+, CD163+ or CD163+TIM3+ tumor-associated macrophages. In certain embodiments, the regulatory T cells are CD4+FOXP3+ regulatory T cells. In certain embodiments, the three cell-structures are detected in the stroma of the tumor. In certain embodiments, the three-cell structures are detected in the perivascular space of the tumor (e.g., within 50-100 μm, preferably within 50 μm, of a tumor blood vessel). In certain embodiments, the three-cell structures are detected in the perivascular stroma of the tumor. In some embodiments, a density of 5 or more three-cell structures per mm² in the perivascular space of the tumor indicates that the tumor will not respond to the anti-cancer therapy. In some embodiments, a density of 10 or more, 15 or more, or 20 or more three-cell structures per mm² in the perivascular space of the tumor indicates that the tumor will not respond, or is less likely to respond, to the anti-cancer therapy. In some embodiments, a two-fold, three-fold, four-fold, five-fold, six-fold, or seven-fold increase in the number of three-cell structures per mm² in the perivascular space of the tumor indicates that the tumor will not respond, or is less likely to respond, to the anti-cancer therapy. In certain preferred embodiments, a four-fold or a five-fold increase in the number of three-cell structures per mm² in the perivascular space of the tumor indicates that the tumor will not respond, or is less likely to respond, to the anti-cancer therapy.

In some embodiments, the methods described herein can be used for determining the prognosis of a cancer. For example, such a method may comprise steps of detecting in a tumor sample the presence of three-cell structures comprising a T cell within 100 μm of (for example, within 50 μm, 20 μm, 15 μm, 10 μm, 5 μm, or less than 5 μm of, and preferably in direct contact with both) an immunosuppressive tumor-associated macrophage and an immunosuppressive regulatory T cell; calculating the density or number of the three-cell structures in the tumor sample; and determining the prognosis of the cancer, wherein a higher density or number of the three-cell structures in the tumor sample indicates a worse prognosis.

In some embodiments, the methods described herein can be used for determining the invasiveness of a tumor. For example, such a method may comprise steps of detecting in a tumor sample the presence of three-cell structures comprising a T cell within 100 μm of (for example, within 50 μm, 20 μm, 15 μm, 10 μm, 5 μm, or less than 5 μm of, and preferably in direct contact with) an immunosuppressive tumor-associated macrophage and an immunosuppressive regulatory T cell; calculating the density or number of the three-cell structures in the tumor sample; and determining the invasiveness of the tumor, wherein the invasiveness of the tumor increases as the density or number of the three-cell structures in the tumor sample increases.

In another aspect, the present disclosure provides methods of treating a tumor in a subject comprising the steps of detecting in a sample of the tumor taken from the subject the presence of three-cell structures comprising a T cell within 100 μm (for example, within 50 μm, 20 μm, 15 μm, 10 μm, 5 μm, or less than 5 μm of, and preferably in direct contact with) of an immunosuppressive tumor-associated macrophage and an immunosuppressive regulatory T cell; calculating the density or number of the three-cell structures in the tumor sample; and administering a treatment to the subject if the density or number of the three-cell structures in the tumor sample is above a baseline threshold. In certain embodiments, the treatment comprises administering an anti-cancer agent, surgery, and/or radiation therapy. In some embodiments, the treatment does not comprise administering an immunotherapy. In certain embodiments, the treatment does not comprise administering a T cell-based immunotherapy. In some embodiments, the T cells in the three-cell structures may be within 90 μm, 80 μm, 70 μm, 60 μm, 50 μm, 40 μm, 30 μm, 20 μm, 15 μm, 10 μm, 5 μm, or less than 5 μm from, or in direct contact with (preferably within 20 μm, 15 μm, 10 μm, 5 μm, or less than 5 μm from, or in direct contact with) the immunosuppressive tumor-associated macrophages and regulatory T cells. In some embodiments, the three-cell structures comprise T cells in direct contact with immunosuppressive tumor-associated macrophages and regulatory T cells. In some embodiments, the T cells are non-functional CD8+ T cells (e.g., inactive PD1−LAG3−CD8+ T cells and/or exhausted PD1+LAG3+CD8+ T cells). In certain embodiments, the T cells are PD1−LAG3−CD3+CD8+ T cells. In certain embodiments, the tumor-associated macrophages are CD68+, CD68+TIM3+, CD163+, or CD163+TIM3+ tumor-associated macrophages. In certain embodiments, the regulatory T cells are CD4+FOXP3+ regulatory T cells. In certain embodiments, the three cell-structures are detected in the stroma of the tumor. In certain embodiments, the three-cell structures are detected in the perivascular space of the tumor (e.g., within 50-100 μm of a tumor blood vessel, preferably within 50 μm of a tumor blood vessel). In certain embodiments, the three-cell structures are detected in the perivascular stroma of the tumor.

In another aspect, the present disclosure provides methods of treating a tumor in a subject comprising the steps of detecting in a sample of the tumor taken from the subject the presence of three-cell structures comprising a T cell within 100 μm of (for example, within 50 μm, 20 μm, 15 μm, 10 μm, 5 μm, or less than 5 μm of, and preferably in direct contact with) an immunosuppressive tumor-associated macrophage and an immunosuppressive regulatory T cell; calculating the density or number of the three-cell structures in the tumor sample; inhibiting the immunosuppressive tumor-associated macrophages and/or the immunosuppressive regulatory T cells in the subject if they are present in the tumor sample; and administering a treatment to the subject. In certain embodiments, the treatment comprises a T cell-based immunotherapy. In some embodiments, the T cells in the three-cell structures may be within 90 μm, 80 μm, 70 μm, 60 μm, 50 μm, 40 μm, 30 μm, 20 μm, 15 μm, 10 μm, 5 μm, or less than 5 μm from, or in direct contact with (preferably within 20 μm, 15 μm, 10 μm, 5 μm, or less than 5 μm from) the immunosuppressive tumor-associated macrophages and regulatory T cells. In some embodiments, the three-cell structures comprise T cells in direct contact with immunosuppressive tumor-associated macrophages and regulatory T cells. In some embodiments, the T cells are non-functional CD8+ T cells (e.g., inactive PD1−LAG3−CD8+ T cells and/or exhausted PD1+LAG3+CD8+ T cells). In certain embodiments, the T cells are PD1−LAG3−CD3+CD8+ T cells. In certain embodiments, the tumor-associated macrophages are CD68+, CD68+TIM3+, CD163+, or CD163+TIM3+ tumor-associated macrophages. In certain embodiments, the regulatory T cells are CD4+FOXP3+ regulatory T cells. In certain embodiments, the three cell-structures are detected in the stroma of the tumor. In certain embodiments, the three-cell structures are detected in the perivascular space of the tumor (e.g., within 50-100 μm, preferably within 50 μm) of a tumor blood vessel). In certain embodiments, the three-cell structures are detected in the perivascular stroma of the tumor.

In another aspect, the present disclosure provides kits for predicting the responsiveness of a tumor (and/or determining the chance of a tumor not responding) to an anti-cancer therapy (e.g., a T cell-based immunotherapy). In some embodiments, the kit comprises agents for detecting a three-cell structure in a tumor sample, wherein the three-cell structure comprises a T cell within 100 μm (for example, within 50 μm, 20 μm, 15 μm, 10 μm, 5 μm, or less than 5 μm (in particular in direct contact with)) of an immunosuppressive tumor-associated macrophage and an immunosuppressive regulatory T cell. In some embodiments, the T cells in the three-cell structures may be within 90 μm, 80 μm, 70 μm, 60 μm, 50 μm, 40 μm, 30 μm, 20 μm, 15 μm, 10 μm, 5 μm, or less than 5 μm from, or in direct contact with (preferably within 20 μm, 15 μm, 10 μm, 5 μm, or less than 5 μm from, or in direct contact with) the immunosuppressive tumor-associated macrophages and regulatory T cells. In some embodiments, the three-cell structures comprise T cells in direct contact with immunosuppressive tumor-associated macrophages and regulatory T cells. In some embodiments, the T cells are non-functional CD8+ T cells (e.g., inactive PD1−LAG3−CD8+ T cells and/or exhausted PD1+LAG3+CD8+ T cells). In certain embodiments, the T cells are PD1−LAG3−CD3+CD8+ T cells. In certain embodiments, the tumor-associated macrophages are CD68+, CD68+TIM3+, CD163+, or CD163+TIM3+ tumor-associated macrophages. In certain embodiments, the regulatory T cells are CD4+FOXP3+ regulatory T cells. In certain embodiments, the three cell-structures are present in the stroma of the tumor. In certain embodiments, the three-cell structures are present in the perivascular space of the tumor (e.g., within 50-100 μm, preferably within 50 μm, of a tumor blood vessel). In certain embodiments, the three-cell structures are present in the perivascular stroma of the tumor.

It should be appreciated that the foregoing concepts, and the additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying drawings.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 provides a schematic of the MultiOmyx® workflow.

FIGS. 2A-2B show quantitative image analysis of a tumor sample following MultiOmyx® analysis. FIG. 2A provides a MultiOmyx® image of a human triple negative breast carcinoma (TNBC). TCI=tumor cell island; S=stroma; bv=CD31+ blood vessel. FIG. 2B shows the boundary of the perivascular (PV) space (50 μm from a vessel within the dashed line) and non-PV areas (>50 μm from a vessel). PANCK, CD31 (white), CD163+TAMs, CD8+ T cells, and FOXP3+ Tregs are present in the region between the dashed line and the CD31+ blood vessel (by). No nuclear stain shown. Bar=50 μm.

FIGS. 3A-3D show the perivascular accumulation of CD163+TIM3+ tumor-associated macrophages (TAMs) (FIGS. 3A-3B) and FOXP3+ Tregs (FIGS. 3C-3D) in untreated and chemotherapy-treated human TNBCs. FIG. 3A and FIG. 3C provide MultiOmyx® images of human TNBCs showing CD163+TIM3+TAMs (FIG. 3A) and CD4+FOXP3+ Tregs (FIG. 3C), both highlighted with arrows. FIG. 3B and FIG. 3D show quantification of these two cell subsets in different tumor regions (i.e., PV and non-PV areas of the stroma vs. tumor cell islands (TCIs)). *P<0.05. Bars=50 μm.

FIGS. 4A-4B show perivascular accumulation of PD1−LAG3−CD3+CD8+ T cells in untreated and chemotherapy-treated human TNBCs. FIG. 4A provides a MultiOmyx® image of a human TNBC showing PD1−LAG3−CD3+CD8+ T cells (arrows). FIG. 4B shows quantification of these cells in different tumor regions (i.e., PV and non-PV areas of the stroma vs. tumor cell islands). *<0.05. Bars=50 μm. This distribution was not seen with PD1+LAG3+CD3+CD8+ T cells.

FIGS. 5A-5C show perivascular accumulation of three-cell structures consisting of PD1−LAG3−CD3+CD8+ T cells in direct contact with two potent immunosuppressive cell types (CD163+TIM3+TAMs and CD4+FOXP3+ Tregs) in the tumor stroma. FIGS. 5A-5B provide MultiOmyx® images (FIG. 5A, low magnification; FIG. 5B, higher magnification) of a human TNBC showing these PV cell trios (highlighted in dashed boxes in FIG. 5B; no DAPI included). FIG. 5C shows quantification of the density of these cell trios in PV and non-PV areas of stroma vs. TCIs. *<0.05. Bars=50 μm.

FIG. 6 shows that PV TAMs can be both TIM3+ or TIM3− in PV immunosuppressive cell trios (ICTs, also referred to as “three-cell structures” throughout the present disclosure). This shows that both TAM subtypes are present in ICTs, with different types of T cells (groups 1-4). FIG. 6 also shows that PV accumulation of ICTs in the stroma (in untreated and/or chemotherapy-treated TNBCs) only occurs with specific combinations of TAMs and T cells (dotted group as shown in the legend provided).

FIG. 7 shows that perivascular ICTs contain mainly non-functional CD8+ T cells. Active CD8+ T cells=PD1+LAG3−CD8+. Non-functional CD8+ T cells include two groups: inactive (PD1−LAG3−CD8+) and exhausted (PD1+LAG3+CD8+).

### DEFINITIONS

Unless defined otherwise, all technical and scientific terms used herein have the meaning commonly understood by a person skilled in the art to which this invention belongs. The following references provide one of skill with a general definition of many of the terms used in this invention: Lawrence et al., Henderson's Dictionary of Biology (16^{th} ed. 2016); Martin et al., Oxford Dictionary of Biology (7^{th} ed. 2015); King et al., A Dictionary of Genetics (8^{th} ed. 2013); Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The terms “tumor” and “neoplasm” are used herein refers to an abnormal mass of tissue wherein the growth of the mass surpasses and is not coordinated with the growth of a normal tissue. A tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. In some embodiments, a tumor is a breast carcinoma. In certain embodiments, a tumor is a triple negative breast carcinoma. In certain embodiments, a tumor is a prostate adenocarcinoma.

Solid tumors are made up of two distinct compartments, referred to herein as the “parenchyma” and the “stroma.” The tumor parenchyma is made up of neoplastic cells. The tumor stroma, induced by the neoplastic cells of the parenchyma, plays a structural and connective role. The stroma is needed for nutritional support and waste removal.

The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva). In certain embodiments, a cancer is breast cancer. In certain embodiments, breast cancer is triple negative breast cancer. In certain embodiments, a cancer is prostate cancer.

Anti-cancer therapies or anti-cancer agents encompass biotherapeutic anti-cancer agents as well as chemotherapeutic agents. Exemplary biotherapeutic anti-cancer agents include, but are not limited to, interferons, cytokines (e.g., tumor necrosis factor, interferon α, interferon γ), vaccines, hematopoietic growth factors, monoclonal serotherapy, immunostimulants and/or immunomodulatory agents (e.g., IL-1, 2, 4, 6, or 12), immune cell growth factors (e.g., GM-CSF) and antibodies (e.g. HERCEPTIN (trastuzumab), T-DM1, AVASTIN (bevacizumab), ERBITUX (cetuximab), VECTIBIX (panitumumab), RITUXAN (rituximab), BEXXAR (tositumomab)). Exemplary chemotherapeutic agents include, but are not limited to, anti-estrogens (e.g. tamoxifen, raloxifene, and megestrol), LHRH agonists (e.g. goscrclin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonuclotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and Thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca²⁺ ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TK1258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genentech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine. In some embodiments, an anti-cancer therapy is an immunotherapy (e.g., a T cell-based immunotherapy). In certain embodiments, an anti-cancer therapy includes a surgery and/or radiation treatment.

An “immunotherapy” or “immunotherapeutic agent” is a therapeutic agent that treats a disease through activation or suppression of the immune system. Immunotherapies are frequently used to treat, for example, various cancers and tumors by artificially stimulating the immune system and improving its natural ability to fight the cancer. Cancer immunotherapy often exploits the fact that cancer cells often have tumor antigens bound to their surface that can be exploited to mark the cancer cells with immunotherapeutic antibodies for the immune system to inhibit or kill. In certain embodiments, an immunotherapy is a T cell-based immunotherapy. T cell-based immunotherapies include, but are not limited to, adoptive cell transfer of tumor-infiltrating lymphocytes, genetically engineered T cells (e.g., CAR-T cell therapies), and immune checkpoint inhibitor antibodies.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In some embodiments, the non-human animal is a mammal (e.g., primate (e.g., cynomolgus monkey or rhesus monkey) or mouse). The term “patient” refers to a subject in need of treatment of a disease. In some embodiments, the subject is human. In some embodiments, the patient is human. The human may be a male or female at any stage of development. A subject or patient “in need” of treatment of a disease or disorder (e.g., a tumor or other form of cancer) includes, without limitation, those who exhibit any risk factors or symptoms of a disease or disorder. Such risk factors or symptoms may be, for example and without limitation, any of those associated with cancer or development of a tumor.

The term “sample” refers to any sample including tissue samples (including tumor samples, e.g., a biopsy of a tumor); cell samples; or cell fractions, fragments, or organelles. In some embodiments, a sample is a tumor sample taken from a subject, for example, a biopsy of a breast carcinoma. In certain embodiments, a sample is a sample from a triple negative breast carcinoma. In certain embodiments, a sample is a sample from a prostate adenocarcinoma.

The terms “treatment,” “treat,” and “treating” refer to reversing, alleviating, delaying the onset of, or inhibiting the progress of a disease described herein (e.g., a tumor or some other form of cancer). In some embodiments, treatment may be administered after one or more signs or symptoms of the disease have developed or have been observed (e.g., prophylactically, or upon suspicion or risk of disease). In other embodiments, treatment may be administered in the absence of signs or symptoms of the disease. For example, treatment may be administered to a susceptible subject prior to the onset of symptoms (e.g., in light of a history of symptoms in the subject, or family members of the subject). Treatment may also be continued after symptoms have resolved, for example, to delay or prevent recurrence. In some embodiments, treatment may be administered after determining the presence of specific cell types or cell structures (e.g., three-cell structures as described herein) in specific quantities in a tumor sample associated with a disease (e.g., triple negative breast carcinoma) using the methods disclosed herein. In certain embodiments, the treatment is an anti-cancer therapeutic, surgery, and/or radiation therapy. In some embodiments, the treatment is an immunotherapy. In certain embodiments, the treatment is a T cell-based immunotherapy. In certain embodiments, the treatment is not a T cell-based immunotherapy.

The terms “administer,” “administering,” and “administration” refer to implanting, absorbing, ingesting, injecting, inhaling, or otherwise introducing a treatment or therapeutic agent, or a composition of treatments or therapeutic agents, in or on a subject.

### DETAILED DESCRIPTION OF CERTAIN EMBODIMENTS

The aspects described herein are not limited to specific embodiments, systems, compositions, methods, or configurations, and as such can, of course, vary. The terminology used herein is for the purpose of describing particular aspects only and, unless specifically defined herein, is not intended to be limiting.

The present disclosure provides methods for predicting the responsiveness of a tumor (and/or determining the chance of a tumor not responding) to an anti-cancer therapy (e.g., an immunotherapy, such as a T cell-based immunotherapy) based on the presence, density, number, and/or location of certain three-cell structures (i.e., immunosuppressive cell trios (ICTs) as described herein). Also provided herein are methods for determining the prognosis and/or invasiveness of a tumor. The present disclosure also encompasses methods for treating a tumor, determining the treatment for an individual, and stratifying patients into groups who will or will not benefit from an anti-cancer therapy (e.g., a T cell-based immunotherapy). Kits for performing the methods disclosed herein are also contemplated by the present disclosure.

### Methods for Determining Treatments, Prognosis, and Invasiveness of Tumors

In one aspect, the present disclosure provides methods for predicting the responsiveness of a tumor (and/or determining the chance of a tumor not responding) to an anti-cancer therapy. In some embodiments, the methods comprise the steps of detecting in a tumor sample the presence of three-cell structures (i.e., immunosuppressive cell trios (ICTs) comprising a T cell within 100 μm (for example, within 50 μm, 20 μm, 15 μm, 10 μm, 5 μm, or less than 5 μm (in particular in direct contact with)) of an immunosuppressive tumor-associated macrophage and an immunosuppressive regulatory T cell; calculating the density or number of the three-cell structures in the tumor sample; and determining the chance of the tumor not responding to the anti-cancer therapy, wherein the chance increases as the density (e.g., number of three-cell structure or fluorescence intensity per mm² in a tumor sample) or number of the three-cell structures in the tumor sample increases. The anti-cancer therapy may be an immunotherapy. In certain embodiments, the anti-cancer therapy is a T cell-based immunotherapy. For example, in a tumor where there is a high abundance of T cells (e.g., a higher abundance than that found in a tumor that is responsive to a T cell-based immunotherapy) within 100 μm (for example, within 50 μm, 20 μm, 15 μm, 10 μm, 5 μm, or less than 5 μm (in particular in direct contact with)) of immunosuppressive tumor-associated macrophages and regulatory T cells, the efficacy of a T cell-based immunotherapy may be reduced as it interacts with the immunosuppressive cell types while entering the tumor. In some embodiments, such a method may be used to stratify subjects into groups who will benefit from treatment with an immunotherapeutic agent (e.g., a T cell-based immunotherapy), and those who will not benefit. In some embodiments, such a method may be used for determining a treatment for a subject who has been diagnosed with cancer (e.g., breast cancer such as triple negative breast cancer and other breast cancers described herein, or prostate cancer).

T cell-based immunotherapies include, but are not limited to, adoptive cell transfer of tumor-infiltrating lymphocytes, genetically engineered T cells (e.g., CAR-T cell therapies, which are modified to add a chimeric antigen receptor (CAR) that specifically recognizes cancer cells), and immune checkpoint inhibitor antibodies (including, for example, anti-PD-L1 antibodies such as atezolizumab, avelumab, and durvalumab, anti-PD-1 antibodies such as pembrolizumab nivolumab, and cemiplimab, anti-CTLA-4 antibodies such as ipilimumab, and anti-LAG-3 antibodies such as relatlimab).

The three-cell structures described herein may be present in any part of the tumor or extra-tumoral tissue. In some embodiments, the three cell-structures are present within the tumor parenchyma. In certain embodiments, the three cell-structures are detected in the stroma of the tumor. In certain embodiments, the three-cell structures are detected in the perivascular space of the tumor (e.g., the space around one or more blood vessels in a tumor, for example, the space within 50 μm of a blood vessel). In certain embodiments, the three-cell structures are detected in the perivascular stroma of the tumor (e.g., the tumor stroma that is within the perivascular space of the tumor). For example, the three-cell structures may be detected within 150 μm, within 100 μm, within 90 μm, within 80 μm, within 70 μm, within 60 μm, or within 50 μm (and preferably within 50 μm, within 40 μm, within 30 μm, within 20 μm, within 15 μm, within 10 μm, within 5 μm, within less than 5 μm, or in direct contact with) of a tumor blood vessel. In some embodiments, the three-cell structures are detected both within the tumor parenchyma and in the stroma, but at varying densities. For example, the three-cell structures may be detected at a greater density or number in the stroma than in the tumor parenchyma, or at a greater density in the perivascular space of the tumor than in other tumor spaces (e.g., at 2 times the density or number, 5 times the density or number, 10 times the density or number, 15 times the density or number, 20 times the density or number, 25 times the density or number, 30 times the density or number, 35 times the density or number, 40 times the density or number, 45 times the density or number, 50 times the density or number, or more than 50 times the density or number). In certain embodiments, the three-cell structures are detected in the stroma but are not detected within the tumor parenchyma. The three-cell structures may also be detected in thin layers of perivascular space that run between cancer parenchyma. In such a space, the three-cell structures are crowded together and in much greater proximity to the tumor parenchyma compared to in the stroma.

In some embodiments, the T cells in the three-cell structures are within 100 μm (for example, within 50 μm, 20 μm, 15 μm, 10 μm, 5 μm, or less than 5 μm, or in direct contact with) from the immunosuppressive tumor-associated macrophages (TAMs) and the immunosuppressive regulatory T cells (Tregs). The T cells may also be closer than 100 μm to the TAMs and Tregs. In some embodiments, the T cells may be closer than 20 μm to the TAMs and Tregs. For example, the T cells in the three-cell structures may be within 90 μm, 80 μm, 70 μm, 60 μm, 50 μm, 40 μm, 30 μm, 20 μm, 15 μm, 10 μm, 5 μm, or less than 5 μm, or in direct contact with from the immunosuppressive TAMs and Tregs. In some embodiments, the T cells in the three-cell structures may be within 15 μm, 10 μm, 5 μm, or less than 5 μm from, or in direct contact with the immunosuppressive TAMs and/or Tregs. In some embodiments, the three-cell structures comprise T cells in direct contact with an immunosuppressive TAM. In some embodiments, the three-cell structures comprise T cells in direct contact with an immunosuppressive Treg. In certain embodiments, the three-cell structures comprise T cells in direct contact with both immunosuppressive TAMs and Tregs. Individual three-cell structures within a single tumor sample can comprise varying distances between the cells involved in each structure. For example, in a single tumor sample, some of the three-cell structures may involve direct contact between the three cells while in others, the cells are within 100 μm, 90 μm, 80 μm, 70 μm, 60 μm, 50 μm, 40 μm, 30 μm, 20 μm, 15 μm, 10 μm, 5 μm, or less than 5 μm from one another. In some embodiments, the cells in the three-cell structures are preferably within 20 μm, 15 μm, 10 μm, 5 μm, or less than 5 μm from one another, or in direct contact with one another. In instances where the three cells are separated from one another by some distance and are not in direct contact with one another, the cells may be interacting, or have interacted, or be about to interact, through secreted factors the migrate through the intercellular space from one cell to another. Whether any of the cells are in direct contact may be determined during the step of detecting in the methods described herein (e.g., following antibody staining and fluorescence microscopy, whether or not the cells are in direct contact with one another may be determined from the microscopy images obtained).

In some embodiments, the methods provided herein comprise a step of determining the density or number of the three-cell structures in the tumor sample, which may then be used to determine the responsiveness of the tumor to an anti-cancer therapy, such as a T cell-based immunotherapy. In some embodiments, a density of 5 or more three-cell structures per mm² in the perivascular space of the tumor indicates that the tumor will not respond, or is less likely to respond, to the anti-cancer therapy. In some embodiments, a density of 10 or more, 15 or more, or 20 or more three-cell structures per mm² in the perivascular space of the tumor indicates that the tumor will not respond, or is less likely to respond, to the anti-cancer therapy. In some embodiments, a density of 2 or fewer (e.g., 2, 1, or 0) three-cell structures per mm² in the perivascular space of the tumor indicates that the tumor will respond, or is more likely to respond, to the anti-cancer therapy. In some embodiments, a two-fold, three-fold, four-fold, five-fold, six-fold, or seven-fold increase in the number of three-cell structures per mm² in the perivascular space of the tumor indicates that the tumor will not respond, or is less likely to respond, to the anti-cancer therapy. In certain preferred embodiments, a four-fold or a five-fold increase in the number of three-cell structures per mm² in the perivascular space of the tumor indicates that the tumor will not respond, or is less likely to respond, to the anti-cancer therapy. It should be appreciated that the density of three-cell structures or fold-change in the density of three-cell structures which indicates that a tumor will not respond to an anti-cancer therapy may vary for different types of cancer, and/or between patients, and that a person of ordinary skill in the art would readily be able to determine the appropriate density and/or fold-change thresholds for a particular cancer or type of tumor (e.g., by testing approximately 20 images of a tumor sample from a patient, in approximately 10-30 regions of interest in the tumor, using the methods described in the Examples and throughout the present disclosure).

In some embodiments, the tumor can be from any cancer (e.g., any cancer disclosed in the National Cancer Institute (NCI) Dictionary of Cancer Terms (21.02d, published Mar. 6, 2021)). In some embodiments, the methods described herein may be performed on any solid tumor (i.e., a solid tumor of any cancer type). In some embodiments, the cancer is any cancer that is associated with or has a perivascular space within the tumor structure. In some embodiments, the cancer is any cancer associated with the presence of any of the cells in the three-cell structures described herein (e.g., TAMs, Tregs, and cytotoxic T cells). In some embodiments, the cancer is any cancer associated with the presence of a CD163+TAM, a TIM3+TAM, a TIM3−TAM, and/or a CD68+TAM (e.g., a CD163+TIM3+TAM, a CD163+TIM3−TAM, a CD68+TIM3+TAM, and/or a CD68+TIM3−TAM). In some embodiments, the cancer is any cancer associated with the presence of a CD4+Treg and/or a FoxP3+Treg (e.g., a CD4+FoxP3+Treg). In some embodiments, the cancer is any cancer associated with the presence of a CD8+ cytotoxic T cell, a PD1+ cytotoxic T cell, a PD1-cytotoxic T cell, a LAG3+ cytotoxic T cell, and/or a LAG3− cytotoxic T cell, (e.g., a CD8+PD1−LAG3− cytotoxic T cell, a CD8+PD1+LAG3− cytotoxic T cell, a CD8+PD1-LAG3+ cytotoxic T cell, and/or a CD8+PD1+LAG3+ cytotoxic T cell). Cancers associated with the presence of T cells and macrophages (including in the perivascular space) are known in the art and are described, for example, in Thorsson et al., The immune landscape of cancer. Immunity 2018, 48, 812-830; Galli, F. et al. Relevance of immune cell and tumor microenvironment imaging in the new era of immunotherapy. J. Exp. Clin. Cancer Res. 2020, 39, 89; and Baer, C. et al. Reciprocal interactions between endothelial cells and macrophage in angiogenic vascular niches. Exp. Cell Res. 2013, 319(11), 1626-34, each of which is incorporated herein by reference.

In some embodiments, the tumor is a breast cancer tumor (e.g., a triple negative breast cancer, ductal carcinoma, lobular carcinoma, inflammatory breast cancer, metastatic breast cancer, paget disease of the breast, angiosarcoma, or phyllodes tumor. In certain embodiments, the tumor is a breast cancer tumor. In certain embodiments, breast cancer is triple negative breast cancer. In certain embodiments, breast cancer comprises ductal carcinoma. In certain embodiments, breast cancer comprises lobular carcinoma. In certain embodiments, breast cancer comprises inflammatory breast cancer. In certain embodiments, breast cancer comprises metastatic breast cancer. In certain embodiments, breast cancer comprises paget disease of the breast. In certain embodiments, breast cancer comprises angiosarcoma. In certain embodiments, breast cancer comprises a phyllodes tumor. In certain embodiments, a tumor is a prostate cancer tumor. In certain embodiments, the tumor is a melanoma.

In some embodiments, the step of detecting comprises performing immunofluorescence. In certain embodiments, the step of detecting comprises performing MultiOmyx® analysis (e.g., as described in the Examples section below, as well as in Gerdes, M. J. et al., Highly multiplexed single-cell analysis of formalin-fixed, paraffin-embedded cancer tissue. Proc. Natl. Acad. Sci. USA 2013, 110(29), 11982, which is incorporated herein by reference). Other suitable methods for performing the step of detecting are known in the art and may include, but are not limited to, those described in U.S. Pat. Nos. 7,741,045 and 8,067,241, both of which are incorporated herein by reference in their entireties. In some embodiments, the step of detecting comprises staining with antibodies directed to different cellular markers (e.g., certain cell surface receptors). In some embodiments, the step of detecting comprises multiple rounds of staining with antibodies directed to different cellular markers (e.g., any of those described herein for use in detecting the Tregs, TAMs, and T cells of the three-cell structures disclosed herein). In some embodiments, two or more, three or more, four or more, five or more, six or more, seven or more, eight or more, nine or more, or ten or more rounds of antibody staining are performed. In some embodiments, each antibody is conjugated to a fluorescent dye (for example, a cyanine dye). In some embodiments, imaging (e.g., immunofluorescence imaging) is performed after each round of antibody staining, thereby allowing observation of the location of the cells expressing each cellular marker. In certain embodiments, a step of quenching is performed between each round of antibody staining, thereby allowing staining with the same antibodies to be performed in the next round of staining and imaging. In some embodiments, the step of quenching comprises dye inactivation chemistry, thereby enabling repeated rounds of staining and quenching (e.g., dye deactivation). Antibodies suitable for use in the presently described methods include, but are not limited to, anti-CTLA-4 antibodies, anti-CD56 antibodies, anti-PanCK antibodies, anti-CD66b antibodies, anti-SMA antibodies, anti-LAG-3 antibodies, anti-CD3 antibodies, anti-arginase antibodies, anti-CD4 antibodies, anti-CD31 antibodies, anti-CD8 antibodies, anti-PD-L1 antibodies, anti-CD11B antibodies, anti-FoxP3 antibodies, anti-CD68 antibodies, anti-CXCR4 antibodies, anti-PD-1 antibodies, anti-TIM3 antibodies, and anti-CD163 antibodies. In some embodiments, preferred antibodies for use in detecting the three-cell structures described herein include anti-CD4 and anti-FoxP3 antibodies (e.g., for detecting the immunosuppressive regulatory T cells of the three-cell structures), anti-CD8, anti-PD1, and anti-LAG3 antibodies (e.g., to detect the cytotoxic T cells of the three-cell structures), and anti-CD163, anti-TIM3, and anti-CD68 antibodies (e.g., for detecting the TAMs of the three-cell structures). In certain embodiments, the immunosuppressive regulatory T cells are detected using anti-CD4 and anti-FoxP3 antibodies. In certain embodiments, the cytotoxic T cells are detected using anti-CD8, anti-PD1, and anti-LAG3 antibodies. In certain embodiments, the TAMs are detected using anti-CD163 and anti-TIM3 antibodies. In certain embodiments, the TAMs are detected using anti-CD68 and anti-TIM3 antibodies. In some embodiments, the step of detecting comprises staining with two antibodies at once. In certain embodiments, the step of detecting comprises staining with two antibodies at once, multiple times (e.g., two times, three times, four times, five times, or more than five times).

A person of ordinary skill in the art will readily appreciate that the step of detecting can be performed in any way that allows identification of the three cell types in the three-cell structures and is not limited to those explicitly described herein. For example, any suitable method known in the art for staining cells, observing the phenotypes and/or genotypes of cells, observing the shapes of cells, and/or looking at any other characteristics associated with the cell types could be used in the methods described herein.

The T cells involved in the three-cell structures of the present disclosure may comprise a variety of cell markers. For example, the T cell involved in each individual three-cell structure may be characterized by the expression or lack of expression of one or more characteristic proteins including, but not limited to, certain cell surface receptors. In some embodiments, the T cell is a PD1− T cell. In some embodiments, the T cell is a PD1+ T cell. In some embodiments, the T cell is a LAG3− T cell. In some embodiments, the T cell is a LAG3+ T cell. In some embodiments, the T cell is a CD3+ T cell. In some embodiments, the T cell is a CD8+ T cell. The T cell may also have a combination of any two or more markers. In certain embodiments, the T cell is a PD1−LAG3−CD3+CD8+ T cell. In certain embodiments, the T cell is a PD1−LAG3−CD8+ T cell. In certain embodiments, the T cell is a PD1+LAG3+CD8+ T cell. In certain embodiments, the T cell is a CD8+PD1+LAG3− T cell. In certain embodiments, the T cell is a CD8+PD1+LAG3+ T cell.

Each of the immunosuppressive tumor-associated macrophages involved in the three-cell structures of the present disclosure may also individually be characterized by the presence or absence of specific cell markers (e.g., certain cell surface receptors or other proteins). In some embodiments, the tumor-associated macrophage is a CD163+ tumor-associated macrophage. In some embodiments, the tumor-associated macrophage is a TIM3+ tumor-associated macrophage. In certain embodiments, the tumor-associated macrophage is a CD163+TIM3+ tumor-associated macrophage. In some embodiments, the tumor-associated macrophage is a CD68+ tumor-associated macrophage. In some embodiments, the tumor-associated macrophage is a CD68+TIM3+ tumor-associated macrophage.

Each of the immunosuppressive regulatory T cells involved in the three-cell structures of the present disclosure may also individually be characterized by the presence or absence of specific cell markers (e.g., certain cell surface receptors or other proteins). In some embodiments, the regulatory T cell is a CD4+ regulatory T cell. In some embodiments, the regulatory T cell is a FOXP3+ regulatory T cell. In certain embodiments, the regulatory T cell is a CD4+FOXP3+ regulatory T cell.

The use of various tumor samples in the methods described herein is contemplated by the present disclosure. In some embodiments, the tumor sample is taken from a subject (e.g., a subject who has been diagnosed with cancer). In some embodiments, the tumor sample is taken from a subject who has a solid tumor. In some embodiments, the tumor sample is taken from a subject who has been diagnosed with breast cancer. In certain embodiments, the tumor is a sample from a breast carcinoma. In certain embodiments, the tumor is a sample from a triple negative breast carcinoma. In some embodiments, the tumor sample is taken from a patient who has been diagnosed with prostate cancer. In certain embodiments, the tumor sample has already been treated by chemotherapy (e.g., the tumor sample is taken from a patient who was treated by chemotherapy). In certain embodiments, the tumor sample has not been exposed to chemotherapy.

In some embodiments, the methods described herein can be used for determining the prognosis of a cancer. For example, such a method may comprise steps of detecting in a tumor sample the presence of three-cell structures comprising a T cell within 100 μm (for example, within 50 μm, 20 μm, 15 μm, 10 μm, 5 μm, or less than 5 μm (in particular in direct contact with)) of an immunosuppressive tumor-associated macrophage and an immunosuppressive regulatory T cell; calculating the density or number of the three-cell structures in the tumor sample; and determining the prognosis of the cancer, wherein a higher density or number of the three-cell structures in the tumor sample indicates a worse prognosis. For example, a cancer may have a worse prognosis when a higher density or number of the three-cell structures are present in the tumor sample because the subject from whom the tumor sample was collected is more likely to not respond to a cancer immunotherapy, such as a T cell-based immunotherapy. The T cell, TAM, and Treg may each have the characteristics of those described elsewhere herein (e.g., each cell may individually be characterized by the presence or absence of specific cell markers as described herein).

In some embodiments, the methods described herein can be used for determining the invasiveness of a tumor (i.e., the degree to which a tumor is expected to metastasize). For example, such a method may comprise steps of detecting in a tumor sample the presence of three-cell structures comprising a T cell within 100 μm (for example, within 50 μm, 20 μm, 15 μm, 10 μm, 5 μm, or less than 5 μm (in particular in direct contact with)) of an immunosuppressive tumor-associated macrophage and an immunosuppressive regulatory T cell; calculating the density or number of the three-cell structures in the tumor sample; and determining the invasiveness of the tumor, wherein the invasiveness of the tumor increases as the density or number of the three-cell structures in the tumor sample increases. The T cell, TAM, and Treg may each have the characteristics of those described elsewhere herein (e.g., each cell may individually be characterized by the presence or absence of specific cell markers as described herein).

In one aspect, the present disclosure provides a method for predicting the responsiveness of a tumor to a T cell-based immunotherapy, the method comprising:
detecting in a tumor sample the presence of three-cell structures comprising a T cell in direct contact with an immunosuppressive tumor-associated macrophage and an immunosuppressive regulatory T cell, wherein the T cell is a PD1−LAG3−CD8+ T cell or a PD1+LAG3+CD8+ T cell, the tumor-associated macrophage is a CD163+TIM3+ tumor-associated macrophage, and the T cell is a CD4+FOXP3+ regulatory T cell;
calculating the density of the three-cell structures in the tumor sample (e.g., the number of the three-cell structures observed within the perivascular space of the tumor stroma); and
predicting the responsiveness of the tumor to the T cell-based immunotherapy, wherein the responsiveness decreases as the density of the three-cell structures in the tumor sample increases. The T cell, TAM, and Treg may each have the characteristics of those described elsewhere herein (e.g., each cell may individually be characterized by the presence or absence of specific cell markers as described herein).

In one aspect, the present disclosure provides a method for determining the chance of a tumor not responding to a T cell-based immunotherapy, the method comprising:
detecting in a tumor sample the presence of three-cell structures comprising a T cell in direct contact with an immunosuppressive tumor-associated macrophage and an immunosuppressive regulatory T cell, wherein the T cell is a PD1−LAG3−CD3+CD8+ T cell, the tumor-associated macrophage is a CD163+TIM3+ tumor-associated macrophage, and the T cell is a CD4+FOXP3+ regulatory T cell;
calculating the density of the three-cell structures in the tumor sample; and
determining the chance of the tumor not responding to the T cell-based immunotherapy, wherein the chance increases as the density of the three-cell structures in the tumor sample increases. The T cell, TAM, and Treg may each have the characteristics of those described elsewhere herein (e.g., each cell may individually be characterized by the presence or absence of specific cell markers as described herein).

### Methods for Treating a Tumor

In one aspect, the present disclosure provides methods for treating a subject having a tumor. In some embodiments, such a method comprises detecting in a sample of the tumor taken from the subject the presence of three-cell structures comprising a T cell within 100 μm of (for example, within 50 μm, 20 μm, 15 μm, 10 μm, 5 μm, or less than 5 μm of, and preferably within direct contact with) an immunosuppressive tumor-associated macrophage and an immunosuppressive regulatory T cell; calculating the density or number of the three-cell structures in the tumor sample; and administering a treatment (e.g., any of the nonimmunotherapy-based anti-cancer therapies described herein, including chemotherapeutic agents, radiation therapy, and surgery) to the subject if the density or number of the three-cell structures in the tumor sample is above a baseline threshold (e.g., a threshold determined based on the density or number of the three-cell structures in one or more tumor samples that respond to an anti-cancer immunotherapy, such as a T cell based immunotherapy). In some embodiments, the treatment comprises administering an immunooncology therapy. In some embodiments, the immunooncology therapy targets macrophages and/or T cells. In some embodiments, the treatment comprises administering an anti-cancer agent. In some embodiments, the treatment comprises surgery. In some embodiments, the treatment comprises radiation therapy. In certain embodiments, the treatment comprises one or more of: administering an anti-cancer agent, surgery, and/or radiation therapy. In some embodiments, the treatment does not comprise administering an immunotherapy. In certain embodiments, the treatment does not comprise administering a T cell-based immunotherapy. For example, if it is determined that the subject has a high density or number of the three-cell structures described herein in the perivascular space of the tumor, the subject may be administered a treatment other than a T cell-based immunotherapy (e.g., any of the nonimmunotherapy-based anti-cancer therapies described herein, including chemotherapeutic agents, radiation therapy, and surgery). The T cell, TAM, and Treg may each have the characteristics of those described elsewhere herein (e.g., each cell may individually be characterized by the presence or absence of specific cell markers as described herein). In some embodiments, the T cell is a PD1−LAG3−CD8+ T cell or a PD1+LAG3+CD8+ T cell, the tumor-associated macrophage is a CD163+TIM3+ tumor-associated macrophage, and the T cell is a CD4+FOXP3+ regulatory T cell.

In another aspect, the present disclosure provides methods of treating a tumor in a subject. In some embodiments, such a method comprises detecting in a sample of the tumor taken from the subject the presence of three-cell structures comprising a T cell within 100 μm (for example, within 50 μm, 20 μm, 15 μm, 10 μm, 5 μm, or less than 5 μm (in particular in direct contact with)) of an immunosuppressive tumor-associated macrophage and an immunosuppressive regulatory T cell; calculating the density or number of the three-cell structures in the tumor sample; inhibiting the immunosuppressive tumor-associated macrophages and/or the immunosuppressive regulatory T cells in the subject if they are present in the tumor sample; and administering a treatment to the subject. In certain embodiments, the treatment comprises an immunotherapy. In certain embodiments, the treatment comprises a T cell-based immunotherapy. The T cell, TAM, and Treg may each have the characteristics of those described elsewhere herein (e.g., each cell may individually be characterized by the presence or absence of specific cell markers as described herein). In some embodiments, the T cell is a PD1−LAG3−CD8+ T cell or a PD1+LAG3+CD8+ T cell, the tumor-associated macrophage is a CD163+TIM3+ tumor-associated macrophage, and the T cell is a CD4+FOXP3+ regulatory T cell.

Other therapies and treatments known in the art can also be used in the methods described herein. In some embodiments, combinations of one or more therapies or treatments are used.

### Kits

Also encompassed by the present disclosure are kits. The kits provided may comprise one or more agents described herein (e.g., an antibody for detecting a cell) and a container (e.g., a vial, ampule, bottle, syringe, and/or dispenser package, or another suitable container). In some embodiments, a kit described herein further includes instructions for using the kit.

In one aspect, the present disclosure provides kits for predicting the responsiveness of a tumor (and/or determining the chance of a tumor not responding) to an anti-cancer therapy. In some embodiments, the kit comprises agents for detecting a three-cell structure in a tumor sample, wherein the three-cell structure comprises a T cell within 100 μm (for example, within 50 μm, 20 μm, 15 μm, 10 μm, 5 μm, or less than 5 μm (in particular in direct contact with)) of an immunosuppressive tumor-associated macrophage and an immunosuppressive regulatory T cell. The T cell, TAM, and Treg may each have the characteristics of those described elsewhere herein (e.g., each cell may individually be characterized by the presence or absence of specific cell markers as described herein). In some embodiments, one or more of the agents is capable of detecting the T cell. In some embodiments, one or more of the agents is capable of detecting the immunosuppressive tumor-associated macrophage. In some embodiments, one or more of the agents is capable of detecting the immunosuppressive regulatory T cell. In certain embodiments, the one or more agents are selected from the group consisting of an anti-CTLA-4 antibody, an anti-CD56 antibody, an anti-PanCK antibody, an anti-CD66b antibody, an anti-SMA antibody, an anti-LAG-3 antibody, an anti-CD3 antibody, an anti-arginase antibody, an anti-CD4 antibody, an anti-CD31 antibody, an anti-CD8 antibody, an anti-PD-L1 antibody, an anti-CD11B antibody, an anti-FoxP3 antibody, an anti-CD68 antibody, an anti-CXCR4 antibody, an anti-PD-1 antibody, an anti-TIM3 antibody, and an anti-CD163 antibody. In some embodiments, the kit comprises suitable agents for staining a tumor sample with two antibodies at once. In certain embodiments, the kit comprises suitable agents for staining a tumor sample with two antibodies at once, multiple times (e.g., two times, three times, four times, five times, or more than five times). In some embodiments, the kit further comprises an anti-cancer therapy (e.g., any of those described herein, or any anti-cancer therapy known in the art). In some embodiments, the anti-cancer therapy is an immunotherapy. In certain embodiments, the immunotherapy is a T cell-based immunotherapy.

### EXAMPLES

### Example 1: Perivascular Accumulation of Immunosuppressive Cells in the Stroma of Human Triple Negative Breast Cancer Carcinomas

MultiOmyx® multiplex immunofluorescence coupled to advanced analytics was used to compare the distribution and phenotype of tumor-associated macrophages (TAMs), CD4+ and CD8+ T cells, and CD4+FOXP3+ regulatory T cells (Tregs) and CD56+NK cells in perivascular (PV) vs. non-PV areas in the stroma and tumor cell islands (TCIs) of 40 human triple negative breast carcinomas (TNBCs), 20 of which were from untreated patients and 20 from patients treated with neoadjuvant chemotherapy. It was also investigated whether the distribution was altered after neoadjuvant chemotherapy. Tregs, along with distinct subsets of TAMs and CD4+ and CD8+ T cells, were found to preferentially accumulate in PV areas (i.e., within 50 μm of CD31+ blood vessels), especially in the tumor stroma. CD163+TIM3+TAMs often made direct contact with the abluminal surface of blood vessels in these sites, and at increased numbers in chemotherapy-treated TNBCs. This suggests they may regulate the formation/function of blood vessels in relapsing tumors. In both untreated and chemotherapy-treated tumors, a major subset of CD163+TAMs lacked PDL1 expression and also accumulated preferentially in stromal PV areas, where many made direct contact with PD1-CD4+ or PD1-CD8+ T cells as well as FOXP3+CD4+Tregs. Close contact of these subsets of T cells with two immunosuppressive cell types (i.e., CD163+TAMs and Tregs) may inhibit their subsequent anti-tumor functions. Taken together, the results presented herein show that as T cells extravasate into TNBCs they encounter a high density of at least two immunosuppressive cell types in the perivascular niche. This could lead to T cell inactivation before the T cells migrate further into tumors, thereby reducing anti-tumor immunity. It could also limit the efficacy of cancer immunotherapies mediated by activated T cells. Targeting the mechanism causing these suppressive cells to accumulate and interact in such sites could remove this inhibition.

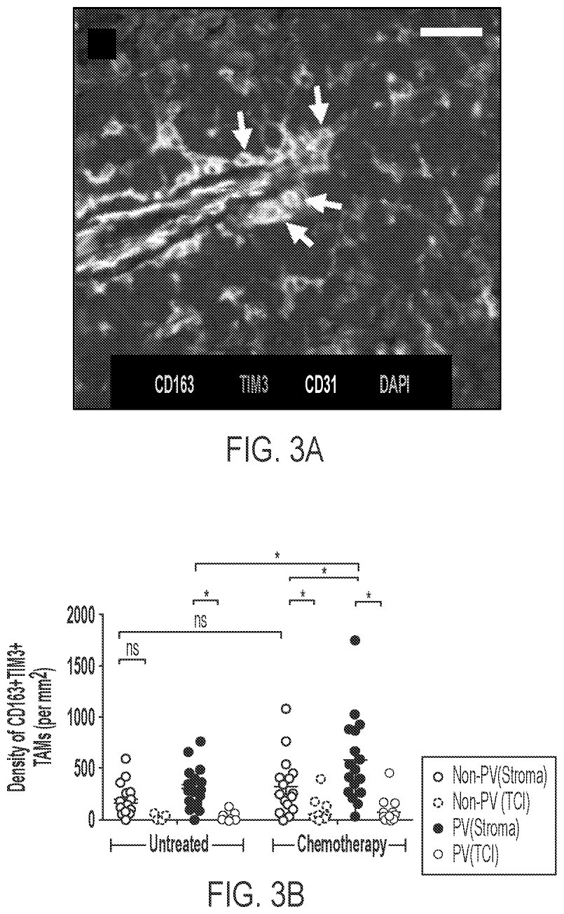

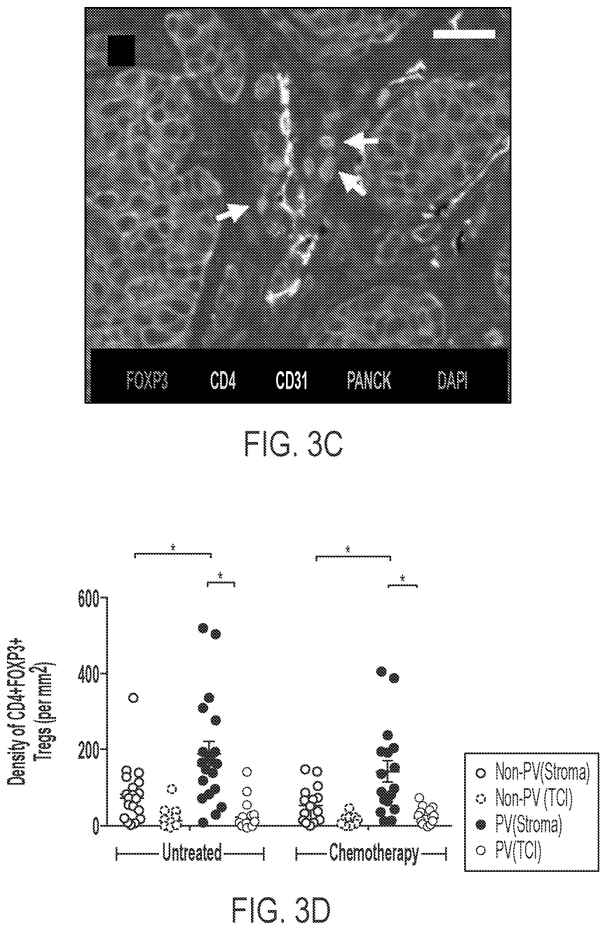

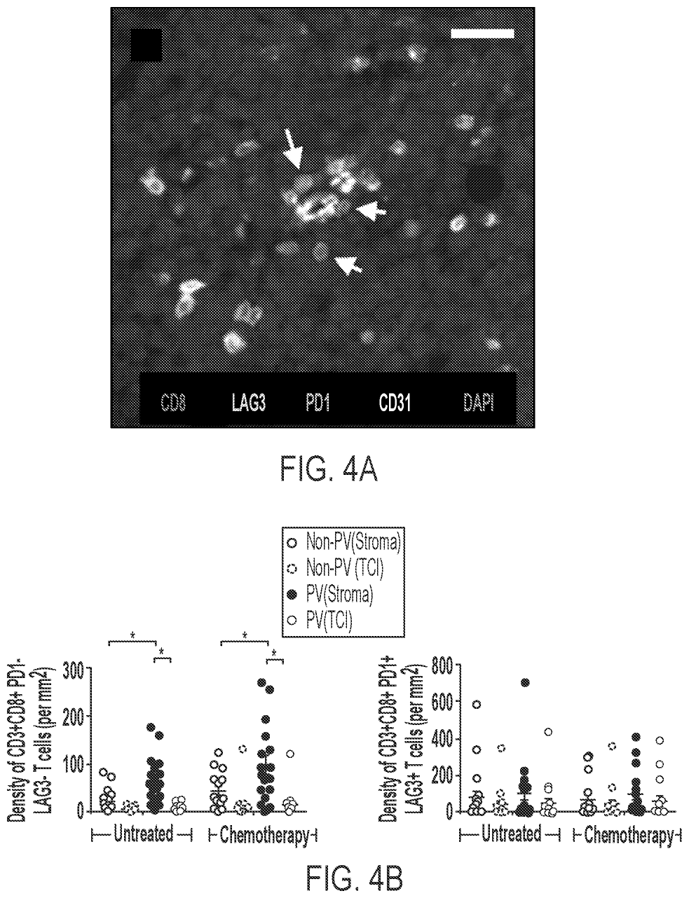

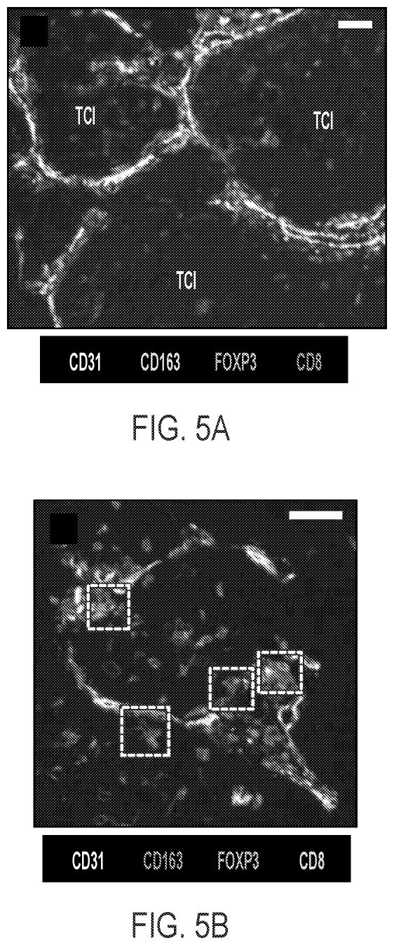

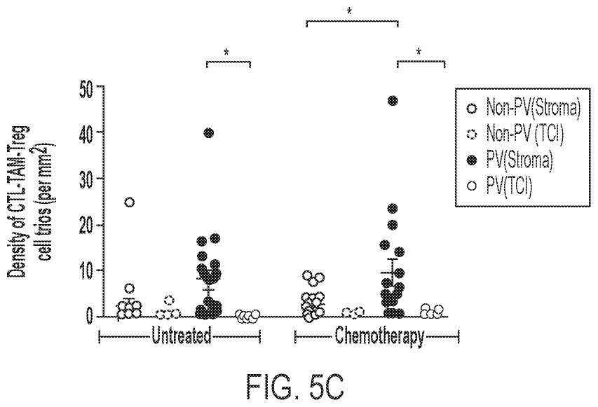

Overall, CD163+TAMs were found to be more abundant throughout the stroma than the TCIs of TNBCs. Around blood vessels in the stroma, these cells were seen to upregulate the negative checkpoint regulator TIM3, especially after chemotherapy (FIG. 3). Both CD4+FOXP3+Tregs and PD1−LAG3−CD3+CD8+ T cells also preferentially accumulated in PV stromal areas (FIGS. 3 and 4). In these PV areas, up to 30% of PD1−LAG3−CD8+ T cells formed direct contact with both CD163+TAMs and Tregs (FIG. 5). Similar 3-cell structures were also formed in PV tumor areas with PD1−LAG3−CD4+ T cells. This intimate contact with two immunosuppressive cell types is highly likely to suppress the function of CD4+ and CD8+ T cells as they cross the vasculature into tumor. The frequency of these PV cell trios could therefore correlate with the efficacy of T cell-based immunotherapies.

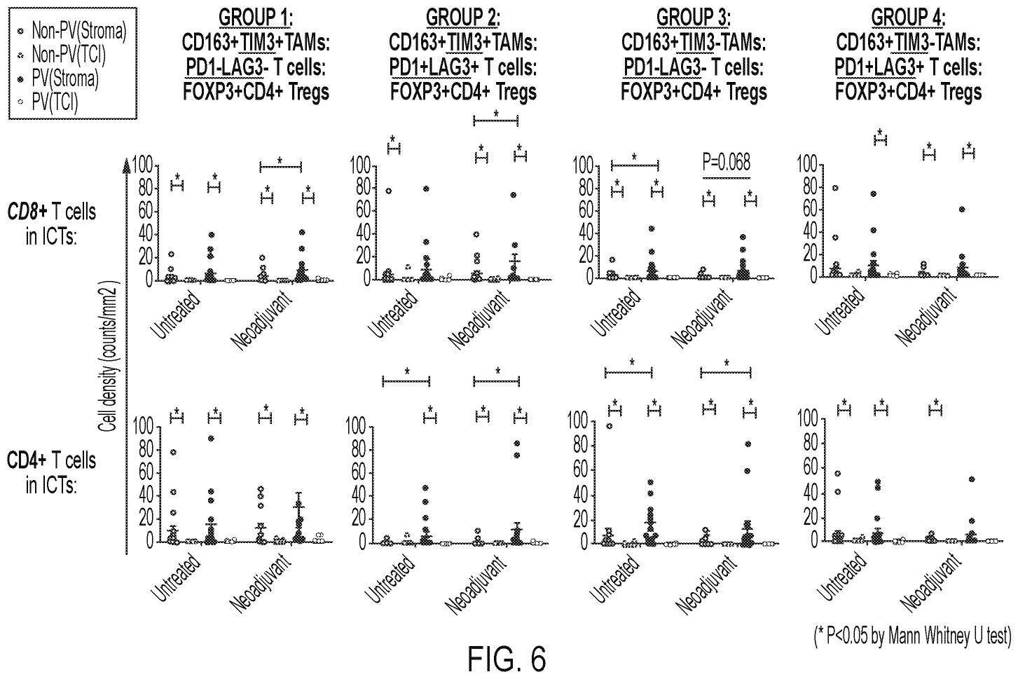

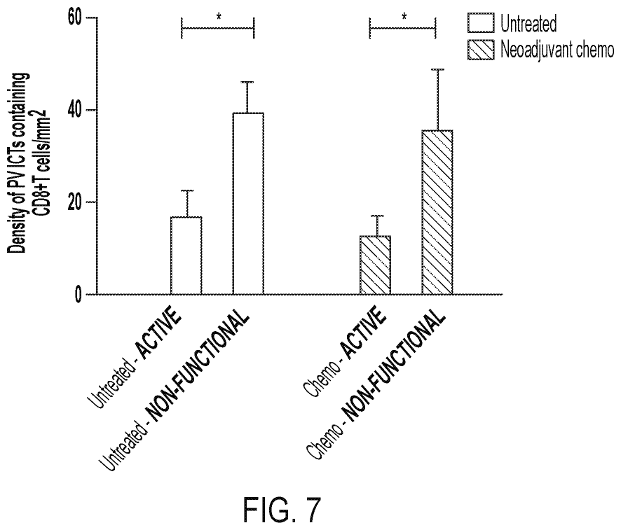

Additionally, PV TAMs can be both TIM3+ or TIM3− in PV ICTs (FIG. 6). The density of stromal PV immunosuppressive cell trios (ICTs, also referred to as “three-cell structures herein) with TIM3+ increases after chemotherapy because the overall density of PV TIM3+TAMs increases as well, and more cells are therefore available to form ICTs. This shows that both TAM subtypes are present in ICTs with different types of T cells (groups 1-4). FIG. 6 also shows that PV accumulation of ICTs in the stroma (in untreated and/or chemotherapy-treated TNBCs) only occurs with specific combinations of TAMs and T cells (dotted group as shown in the legend provided, in the boxes in groups 1-3). This shows that the ICTs that accumulate preferentially around stromal blood vessels in TNBCs are either: (i) CD163+TIM3+TAMs: PD1−LAG3−CD8+ T cells: FOXP3+CD8+Tregs; (ii) CD163+TIM3+TAMs: PD1+LAG3+CD4+ or CD8+ T cells: FOXP3+CD4+Tregs; or (iii) CD163+TIM3-TAMs: PD1−LAG2− T cells: FOXP3+CD4+Tregs. FIG. 7 also shows that the T cells in the ICTs are mainly non-functional CD8+ T cells, and in particular, PD1−LAG3-CD8+ T cells, PD1+LAG3+CD8+ T cells, or a mixture of the two. Therefore, TIM3+TAMs only associate with active PD1+LAG3+ T cells in PV ICTs (where TAMs may need to upregulate TIM3 in order to deactivate the T cells), and TIM3− TAMs only associate with PD1−LAG3− T cells in PV ICTs (where they no longer need their TIM3 as the T cells are already inactive). This shows that the interaction of these cell types in these ICTs displays selectivity.

The markers used to identify the Tregs in the ICTs include CD4+FoxP3+. The markers used to identify the cytotoxic T cells in the ICTs include CD8+PD1−LAG3−, CD8+PD1+LAG3−, CD8+PD1−LAG3+, and CD8+PD1+LAG3+. The markers used to identify the TAMs in the ICTs include CD163+TIM3+, CD163+TIM3−, CD68+TIM3+, and CD68+TIM3−.

The present example describes the detection of the ICTs described herein in a triple negative breast cancer carcinoma, but the inventors contemplate the detection of the ICTs and the use of the methods described herein for determining the responsiveness of a cancer to an anti-cancer therapy in the context of any cancer, in particular any solid tumor, and in particular any cancer that is associated with the presence of a perivascular space. For example, the cancer may be breast cancer (including, for example, any of the types of breast cancer described herein), prostate cancer, or melanoma. The methods described herein are not limited in this respect.

### Methods

MultiOmyx® Staining: MultiOmyx® analysis (described further below, and in Gerdes, M. J. et al. Highly multiplexed single-cell analysis of formalin-fixed, paraffin-embedded cancer tissue. Proc. Natl. Acad. Sci. USA 2013, 110(29), 11982, which is incorporated herein by reference) was performed. Whole FFPE tissue arrays were baked at 65° C. for 1 h. Slides were deparaffinized with xylene, rehydrated by washes of decreasing ethanol concentration, and then processed for antigen retrieval. A two-step antigen retrieval process was adopted to allow antibodies with different antigen retrieval conditions to be used together on the same samples. Samples were then blocked against nonspecific binding with 10% (wt/vol) donkey serum and 3% (wt/vol) BSA in PBS for 1 h at room temperature and stained with DAPI for 15 min. Directly conjugated primary antibodies were diluted in PBS supplemented with 3% (wt/vol) BSA to optimize concentrations and applied for 1 h at room temperature on a Leica Bond III Stainer. In the case of CTLA-4, which was used for primary-secondary antibody staining, samples were incubated with primary CTLA-4 antibody followed by incubation with a species-specific secondary antibody conjugated to cyanine 3 (Cy3).

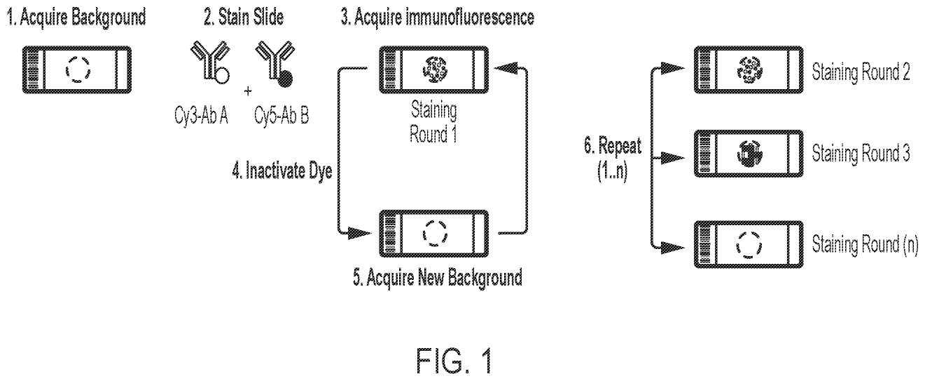

A total of 11 rounds of antibody staining were performed in sequence on the FFPE slides. The MultiOmyx protein immunofluorescence (IF) assay utilizes a pair of directly conjugated cyanine dye-labeled (Cy3, Cy5) antibodies per round of staining. Each round of staining is imaged and followed by dye inactivation chemistry, enabling repeated rounds of staining and deactivation. CTLA-4 and CD56 were stained in round 1, followed by PanCK and CD66b in round 2, SMA and LAG-3 in round 3, CD3 and arginase in round 4, CD4 and CD31 in round 5, CD8 and PD-L1 1 in round 6, CD11b and FoxP3 in round 7, CD68 and CXCR4 in round 8, PD-1 in round 9, TIM-3 in round 10, and CD163 in round 11 (FIG. 1).

After each round of staining with two antibodies, high resolution images were collected from 20 viable PV and non-PV areas in both PanCK-rich areas (TCIs) and PanCK-negative areas (stroma) using a 20× objective on an INCell analyzer 2200 microscope (GE Healthcare Life Sciences). Slides were then washed in PBS/0.3% TritonX-100, and dye inactivation was performed by immersion in an alkaline solution containing H₂O₂ for 15 minutes with gentle agitation at room temperature (Gerdes, M. J. et al. Highly multiplexed single-cell analysis of formalin-fixed, paraffin-embedded cancer tissue. Proc. Natl. Acad. Sci. USA 2013, 110(29), 11982). Slides were washed again in PBS, imaged to check the efficacy of the dye inactivation, and stained with the next round of antibodies.

Antibodies used for MultiOmyx® Multiplexing: Antibodies, by staining order, were mouse anti-CTLA-4 (F-8, Santa Cruz Biotechnology), rabbit anti-CD56 (MRQ-42, Cell Marque), mouse anti-PanCK (cust02300/C5992, eBioScience/Sigma), mouse anti-CD66b (G10F5, BioLegend), mouse anti-SMA (1A4, Sigma), mouse anti-LAG-3 (17B4, LSBio), mouse anti-CD3 (F7.2.38, Dako), rabbit anti-Arginase (EPR6672(B), Abcam), rabbit anti-CD4 (EPR6855, Abcam), mouse anti-CD31 (89C2, Cell Signaling), mouse anti-CD8 (C8/144B, Dako), rabbit anti-PD-L1 (SP142, Abcam), mouse anti-CD11B (238439, R&D Systems), mouse anti-FoxP3 (206D, BioLegend), mouse anti-CD68 (KP1, BioLegend), rabbit anti-CXCR4 (UMB2, Abcam), rabbit anti-PD-1 (EPR48779(2), Abcam), TIM-3 (polyclonal, R&D Systems), and mouse anti-CD163 (EDHu-1, BioRad).

Image Analysis for MultiOmyx®: MultiOmyx® image analysis makes use of illumination-corrected, autofluorescent-subtracted, and registered images from NeoGenomics' multiplexed immunofluorescent process. For analysis in this study, each cell was segmented out using a deep learning algorithm on the DAPI channel and assigned a unique ID. Deep-learning-based classification models were then used to assign a positivity value to each cell for each of the biomarkers in the panel. Using these biomarker positivity values, the phenotype of each cell was determined through co-expression analysis. To prevent the presence of incomplete biomarker information from introducing errors in determining cell phenotypes, a tissue quality mask was applied to remove all cells from the analysis that did not have complete biomarker information for each round of staining, which may happen when tissue tears or tissue folding occurs during the staining process. Once each cell's phenotype was determined, cell cluster analysis was performed at various distances from vessels identified in the samples. Both triads and diads (three or two cells of various phenotypes that form a cluster of cells) were examined within the immediate vicinity of the vessels (touching the vessels), within 50 microns from the vessels, and more than 50 microns from the vessels (see Lapenna, A. et al. Perivascular macrophages in health and disease. Nat. Rev. Immunol. 2018, 18, 689-702, which is incorporated herein by reference).

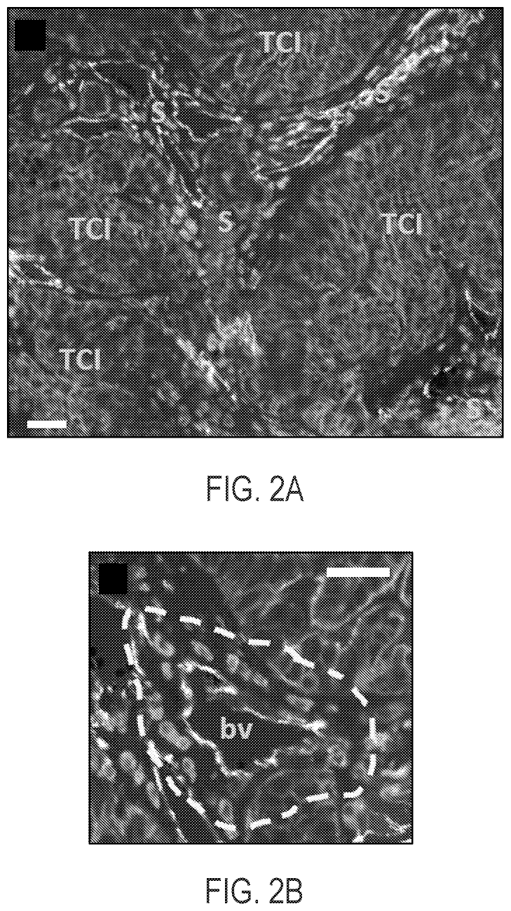

An AI-based advanced analytics platform (NEO Image Analysis) was used to quantify and analyze subsets of immune cell types in TNBCs including algorithms that could differentiate between them in TCIs vs. the tumor stroma (FIG. 2A), and within PV (within 50 μm from a CD31+ blood vessel) or non-PV areas (>50 μm) of these regions (FIG. 2B).

Cells were segmented and tracked through each staining round; deep learning models were used to classify positivity value for each biomarker stain, as well as to classify regions as within TCIs or stroma. Stain co-expression analysis was used to define specific cell phenotypes.

### INCORPORATION BY REFERENCE

The present application refers to various issued patent, published patent applications, scientific journal articles, and other publications, all of which are incorporated herein by reference. The details of one or more embodiments of the invention are set forth herein. Other features, objects, and advantages of the invention will be apparent from the Detailed Description, the Figures, the Examples, and the Claims.

### EQUIVALENTS AND SCOPE

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following claims.

What is claimed is:

1. A method for determining the chance of a tumor not responding to an anti-cancer therapy, the method comprising:
detecting in a tumor sample the presence of three-cell structures comprising a T cell within 100 μm (for example, within 50 μm, 20 μm, 15 μm, 10 μm, 5 μm, or less than 5 μm (in particular in direct contact with)) of an immunosuppressive tumor-associated macrophage and an immunosuppressive regulatory T cell;
calculating the density or number of the three-cell structures in the tumor sample; and
determining the chance of the tumor not responding to the anti-cancer therapy, wherein the chance increases as the density or number of the three-cell structures in the tumor sample increases.

2. A method for predicting the responsiveness of a tumor to an anti-cancer therapy, the method comprising:
detecting in a tumor sample the presence of three-cell structures comprising a T cell within 100 μm (for example, within 50 μm, 20 μm, 15 μm, 10 μm, 5 μm, or less than 5 μm (in particular in direct contact with)) of an immunosuppressive tumor-associated macrophage and an immunosuppressive regulatory T cell;
calculating the density or number of the three-cell structures in the tumor sample; and
predicting the responsiveness of the tumor to the anti-cancer therapy, wherein the responsiveness of the tumor to the anti-cancer therapy decreases as the density or number of the three-cell structures in the tumor sample increases.

3. The method of claim 1 or 2, wherein the anti-cancer therapy is an immunotherapy.

4. The method of any one of claims 1-3, wherein the anti-cancer therapy is a T cell-based immunotherapy (e.g., genetically engineered T cells such as CAR-T cells, or immune checkpoint inhibitors).

5. A method for determining the prognosis of a cancer, the method comprising:
detecting in a tumor sample the presence of three-cell structures comprising a T cell within 100 μm (for example, within 50 μm, 20 μm, 15 μm, 10 μm, 5 μm, or less than 5 μm (in particular in direct contact with)) of an immunosuppressive tumor-associated macrophage and an immunosuppressive regulatory T cell;
calculating the density or number of the three-cell structures in the tumor sample; and
determining the prognosis of the cancer, wherein a higher density or number of the three-cell structures in the tumor sample indicates a worse prognosis.

6. A method for determining the invasiveness of a tumor, the method comprising:
detecting in a tumor sample the presence of three-cell structures comprising a T cell within 100 μm (for example, within 50 μm, 20 μm, 15 μm, 10 μm, 5 μm, or less than 5 μm (in particular in direct contact with)) of an immunosuppressive tumor-associated macrophage and an immunosuppressive regulatory T cell;
calculating the density or number of the three-cell structures in the tumor sample; and
determining the invasiveness of the tumor, wherein the invasiveness of the tumor increases as the density or number of the three-cell structures in the tumor sample increases.

7. A method for determining the chance of a tumor not responding to a T cell-based immunotherapy, the method comprising:
detecting in a tumor sample the presence of three-cell structures comprising a T cell in direct contact with an immunosuppressive tumor-associated macrophage and an immunosuppressive regulatory T cell, wherein the T cell is a PD1−LAG3−CD3+CD8+ T cell or a PD1+LAG3+CD3+CD8+ T cell, the tumor-associated macrophage is a CD163+TIM3+ tumor-associated macrophage, and the T cell is a CD4+FOXP3+ regulatory T cell;
calculating the density of the three-cell structures in the tumor sample; and
determining the chance of the tumor not responding to the T cell-based immunotherapy, wherein the chance increases as the density of the three-cell structures in the tumor sample increases.

8. A method for treating a tumor in a subject, the method comprising:
detecting in a sample of the tumor taken from the subject the presence of three-cell structures comprising a T cell within 100 μm (for example, within 50 μm, 20 μm, 15 am, 10 μm, 5 μm, or less than 5 μm (in particular in direct contact with)) of an immunosuppressive tumor-associated macrophage and an immunosuppressive regulatory T cell;
calculating the density or number of the three-cell structures in the tumor sample; and
administering a treatment to the subject if the density or number of the three-cell structures in the tumor sample is above a baseline threshold.

9. The method of claim 8, wherein the treatment comprises administering an anti-cancer agent, surgery, and/or radiation therapy.

10. The method of claim 8 or 9, wherein the treatment does not comprise administering an immunotherapy.

11. The method of any one of claims 8-10, wherein the treatment does not comprise administering a T cell-based immunotherapy (e.g., genetically engineered T cells such as CAR-T cells, or immune checkpoint inhibitors).

12. A method of treating a tumor in a subject, the method comprising:
detecting in a sample of the tumor taken from the subject the presence of three-cell structures comprising a T cell within 100 μm (for example, within 50 μm, 20 μm, 15 μm, 10 μm, 5 μm, or less than 5 μm (in particular in direct contact with)) of an immunosuppressive tumor-associated macrophage and an immunosuppressive regulatory T cell;
calculating the density or number of the three-cell structures in the tumor sample;
inhibiting the immunosuppressive tumor-associated macrophages and/or the immunosuppressive regulatory T cells in the subject if they are present in the tumor sample; and
administering a treatment to the subject.

13. The method of claim 12, wherein the treatment comprises a T cell-based immunotherapy.

14. The method of any one of claims 1-13, wherein the three-cell structures are detected in the stroma of the tumor.

15. The method of any one of claims 1-14, wherein the three-cell structures are detected in the perivascular space of the tumor (for example, within 50 μm of a tumor blood vessel).

16. The method of any one of claims 1-15, wherein the three-cell structures are detected in perivascular areas of the tumor stroma.

17. The method of any one of claims 1-16, wherein the T cell is within 20 μm, 15 μm, 10 μm, 5 μm, or less than 5 μm of the immunosuppressive tumor-associated macrophage.

18. The method of any one of claims 1-17, wherein the T cell is within 20 μm, 15 μm, 10 μm, 5 μm, or less than 5 μm of the immunosuppressive regulatory T cell.

19. The method of any one of claims 1-18, wherein the T cell is in direct contact with the immunosuppressive tumor-associated macrophage and/or the immunosuppressive regulatory T cell.

20. The method of any one of claims 1-19, wherein the three-cell structures are detected within 50-100 μm (e.g., within 50 μm) of a tumor blood vessel.

21. The method of any one of claims 1-20, wherein the step of detecting comprises performing immunofluorescence.

22. The method of any one of claims 1-21, wherein the step of detecting comprises performing MultiOmyx® analysis.

23. The method of any one of claims 1-22, wherein the step of detecting comprises staining with one or more antibodies to detect one or more cellular markers of each of the T cell, tumor-associated macrophage, and regulatory T cell.

24. The method of claim 23, wherein the one or more antibodies are selected from the group consisting of anti-CD4 antibodies, anti-FoxP3 antibodies, anti-CD8 antibodies, anti-PD1 antibodies, anti-LAG3 antibodies, anti-CD163 antibodies, anti-TIM3 antibodies, and anti-CD68 antibodies.

25. The method of claim 23 or 24, wherein the step of detecting further comprises performing fluorescence microscopy following antibody staining to determine the presence and location of the T cell, tumor-associated macrophage, and regulatory T cell within the tumor sample.

26. The method of any one of claims 1-25, wherein the T cell is a PD1− T cell.

27. The method of any one of claims 1-25, wherein the T cell is a PD1+ T cell.

28. The method of any one of claims 1-27, wherein the T cell is a LAG3− T cell.

29. The method of any one of claims 1-27, wherein the T cell is a LAG3+ T cell.

30. The method of any one of claims 1-29, wherein the T cell is a CD3+ T cell.

31. The method of any one of claims 1-30, wherein the T cell is a CD8+ T cell.

32. The method of any one of claims 1-25, wherein the T cell is a PD1−LAG3−CD8+ T cell.

33. The method of any one of claims 1-25, wherein the T cell is a PD1+LAG3+CD8+ T cell.

34. The method of any one of claims 1-25, wherein the T cell is a CD8+PD1+LAG3− T cell.

35. The method of any one of claims 1-25, wherein the T cell is a CD8+PD1−LAG3+ T cell.

36. The method of any one of claims 1-25, wherein the T cell is a PD1−LAG3− CD3+CD8+ T cell.

37. The method of any one of claims 1-36, wherein the tumor-associated macrophage is a CD163+ tumor-associated macrophage.

38. The method of any one of claims 1-37, wherein the tumor-associated macrophage is a TIM3+ tumor-associated macrophage.

39. The method of any one of claims 1-37, wherein the tumor-associated macrophage is a TIM3− tumor-associated macrophage.

40. The method of any one of claims 1-39, wherein the tumor-associated macrophage is a CD68+ tumor-associated macrophage.

41. The method of any one of claims 1-36, wherein the tumor-associated macrophage is a CD163+TIM3+ tumor-associated macrophage.

42. The method of any one of claims 1-36, wherein the tumor-associated macrophage is a CD163+TIM3− tumor-associated macrophage.

43. The method of any one of claims 1-36, wherein the tumor-associated macrophage is a CD68+TIM3+ tumor-associated macrophage.

44. The method of any one of claims 1-36, wherein the tumor-associated macrophage is a CD68+TIM3− tumor-associated macrophage.

45. The method of any one of claims 1-44, wherein the regulatory T cell is a CD4+ regulatory T cell.

46. The method of any one of claims 1-45, wherein the regulatory T cell is a FOXP3+ regulatory T cell.

47. The method of any one of claims 1-44, wherein the regulatory T cell is a CD4+FOXP3+ regulatory T cell.

48. The method of any one of claims 1-47, wherein the tumor sample is taken from a subject.

49. The method of any one of claims 1-48, wherein the tumor sample is a sample from a breast carcinoma.

50. The method of any one of claims 1-49, wherein the tumor sample is a sample from a triple negative breast carcinoma.

51. The method of any one of claims 1-48, wherein the tumor sample is a prostate cancer tumor sample.

52. The method of claim 8, wherein the baseline threshold is determined based on the density or number of the three-cell structures in one or more tumor samples that respond to a T cell-based anti-cancer immunotherapy.

53. A kit for determining the chance of a tumor not responding to an anti-cancer therapy comprising agents for detecting a three-cell structure in a tumor sample, wherein the three-cell structure comprises a T cell within 100 μm (for example, within 50 μm, 20 μm, 15 μm, 10 μm, 5 μm, or less than 5 μm (in particular in direct contact with)) of an immunosuppressive tumor-associated macrophage and an immunosuppressive regulatory T cell.

54. The kit of claim 53, wherein one or more of the agents is capable of detecting the T cell, one or more of the agents is capable of detecting the immunosuppressive tumor-associated macrophage, and one or more of the agents is capable of detecting the immunosuppressive regulatory T cell.

55. The kit of claim 53 or 54, wherein the one or more agents are selected from the group consisting of an anti-CTLA-4 antibody, an anti-CD56 antibody, an anti-PanCK antibody, an anti-CD66b antibody, an anti-SMA antibody, an anti-LAG-3 antibody, an anti-CD3 antibody, an anti-arginase antibody, an anti-CD4 antibody, an anti-CD31 antibody, an anti-CD8 antibody, an anti-PD-L1 antibody, an anti-CD11B antibody, an anti-FoxP3 antibody, an anti-CD68 antibody, an anti-CXCR4 antibody, an anti-PD-1 antibody, an anti-TIM3 antibody, and an anti-CD163 antibody.

56. The kit of claim 55, wherein the one or more agents are selected from the group consisting of an anti-CD4 antibody, an anti-FoxP3 antibody, an anti-CD8 antibody, an anti-PD1 antibody, an anti-LAG3 antibody, an anti-CD163 antibody, an anti-TIM3 antibody, and an anti-CD68 antibody.

57. The kit of any one of claims 53-56 further comprising an anti-cancer therapy.

58. The kit of claim 57, wherein the anti-cancer therapy is an immunotherapy.

59. The kit of claim 58, wherein the immunotherapy is a T cell-based immunotherapy.
